(19) United States
(12) Patent Application Publication
(10) Pub. No.: US 2026/0150869 A1
(43) Pub. Date: Jun. 4, 2026

(54) A METHOD FOR ENHANCING THE FLAVOR AND/OR THE TEXTURE OF A PLANT-BASED PRODUCT

(71) Applicant: Novozymes A/S, Bagsvaerd (DK)

(72) Inventor: Katja PUDER, Bagsvaerd (DK)

(73) Assignee: Novozymes A/S, Bagsvaerd (DK)

(21) Appl. No.: 19/122,941

(22) PCT Filed: Oct. 25, 2023
(86) PCT No.: PCT/EP2023/079747

§ 371 Date: Apr. 21, 2025

(30) Foreign Application Priority Data
Oct. 28, 2022 (EP) 22204282.2

(51) Int. Cl. A23L 11/65; A23L 11/30; C12N 9/40

(52) U.S. Cl. CPC A23L 11/65; A23L 11/30; C12N 9/2465; C12Y 302/01022

(57) ABSTRACT

The present invention relates to methods, uses and process for enhancing the flavor and/or the texture of a plant-based product, such as food or drinks. The present invention relates to use of one or more alpha galactosidases for enhancing the flavor and/or the texture of a plant-based product. The one or more alpha galactosidases can be added to plant-based products or in the process of preparing of a plant-based product.

### REFERENCE TO SEQUENCE LISTING

This application contains a Sequence Listing in computer readable form. The computer readable form is incorporated herein by reference.

### FIELD OF THE INVENTION

The present invention relates to a method for enhancing the flavor and/or the texture of a plant-based product. The present invention particularly relates to a method for enhancing the flavor and/or the texture of a plant-based product, such as food or drinks, using alpha galactosidases.

### BACKGROUND

Recently, the interest of consumers in healthy foods has been increasing, which has made natural foods and functional foods popular. Among these, plant-based products made from legumes contain good quality protein and plant linoleic acid, and also contain plenty of nutrients such as vitamin E and lecithin.

For example, the amount of soymilk consumed in the form of functional food is increasing year after year. Numerous research has been conducted on plant-based products such as soymilk and the method for producing soymilk. Soymilk is however still not widely accepted as beverage due to soyabean odor, texture, mouthfeel, raw smell, astringency, and so forth specific for soyabeans. Food additives such as whey protein, palatinose, or sugar phosphate may be added to enhance the flavors of soymilk.

Inactivating lipoxygenase by heating and/or altering pH of the aqueous medium in which the seeds are disintegrated is another approach to enhance flavour of soymilk, however these treatments tend to insolubilize the soyabean protein and thereby reduce soymilk yield and make it chalky in the mouthfeel.

Existing methods of making soymilk without a beany flavor need improvement. Thus, there is a need for preparing legume-based products with enhanced properties such as sweetness, decreased astringency, richness, fattiness, sweet aftertaste, mouth feel and texture.

### SUMMARY OF THE CLAIMED INVENTION

The present invention relates to enhancing the flavor and/or the texture of a plant-based product, such as food or drinks, using alpha galactosidases.

In one aspect, the present invention relates to use of one or more alpha galactosidases for enhancing the flavor and/or the texture of a plant-based product, wherein the plant-based product is a food and/or a drink.

In another aspect, the present invention relates to a method for enhancing the flavor and/or the texture of a product, by adding one or more alpha galactosidases to a plant-based material and/or the plant-based product and incubating the plant-based material and/or the plant-based product, wherein the plant-based product is a food and/or a drink.

In yet another aspect, the present invention relates to a process for preparing a plant-based product with enhanced flavor and/or texture, wherein the plant-based product is a food and/or a drink. The process comprises soaking one or more plant material and grinding the one or more plant material to a slurry. One or more alpha galactosidases is added to the slurry. Thereafter, fiber is separated from the slurry to obtain plant material milk base. The one or more alpha galactosidases is added to the plant material milk base to obtain the plant-based product.

### Overview of Sequence Listing

SEQ ID NO:1 is mature amino acid sequence of alpha galactosidase derived from Aspergillus niger.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. For the sake of brevity and/or clarity, well-known functions or constructions may not be described in detail.

As used herein, the term alpha galactosidase means a glycoside hydrolase enzyme that catalyses the reaction of hydrolysis of terminal, non-reducing α-D-galactose residues in α-D-galactosides, including galactose oligosaccharides, galactomannans and galactolipids. For the purpose of the present invention, the alpha galactosidase is an enzyme that breaks down the carbohydrates in plant materials, such as grains, legumes, and cruciferous vegetables, into simpler sugars while making plant-based products.

As used herein, the term beany texture means grainy, with a nutty, and dense aftertaste. For the purpose of present invention, the beany texture is grainy, nutty and dense aftertaste of a plant-based product such as soymilk.

As used herein, the term legume texture means grainy, with a nutty, and dense aftertaste. For the purpose of present invention, the legume texture is grainy, nutty and dense aftertaste of a plant-based product such as soymilk.

As used herein, the term astringency means quality of tasting or smelling dry, acidic, and slightly bitter.

As used herein, the term richness means having a large proportion of flavoursome or fatty ingredients, such as butter or cream. For the purpose of present invention, the richness means flavoursome and creamy plant-based product such as flavoursome and creamy soymilk.

As used herein, the term sweet aftertaste means taste that remains after consuming a sweet product.

As used herein, the term mouthfeel means a distinct taste of food or drink that feels in the mouth.

As used herein, the term “raffinose family oligosaccharides” (RFOs) are α-1, 6-galactosyl extensions of sucrose such as raffinose, stachyose, and verbascose.

As used herein, the term plant-based material means a materials and products that are partially or wholly derived from plants. For the purpose of present invention, the plant-based material means materials and products that are partially or wholly derived from legumes such as soyabean, peas, chickpeas, lentils, fava bean and/or any legumes containing raffinose family oligosaccharides. For example, the plant-based material can be legume base or legume slurry, or legume paste.

As used herein, the term plant material means a part of a plant such as stems, roots, seeds, and leaves. For the purpose of present invention, the plant material means plant seeds, preferably plant seeds of leguminous plant.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the no brief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the no brief option must be specified in the command line. The output of Needle labeled “longest identity” is calculated as follows:

(Identical Deoxyribonucleotides×100)/(Length of Alignment−Total Number of Gaps in Alignment)

While certain aspects of the present disclosure will hereinafter be described with reference to embodiments thereof, it will be understood by those of ordinary skill in the art that various changes in form and details may be made therein without departing from the spirit and scope of the present invention as defined by the claims.

### DETAILED DESCRIPTION OF THE INVENTION

The present invention relates to methods, uses and products for enhancing the flavor and/or the texture of a plant-based product.

In order to provide an enhanced flavor and/or texture to a plant-based product such as soymilk, the inventors identified that on addition of alpha galactosidases to a soymilk or during production of soymilk, enhances the texture and/or taste such as the sweetness, richness, fattiness, sweet aftertaste and/or mouth feel, /or decreases bitterness, astringency and/or feeling of a beany texture.

### Plant-Based Product:

In an embodiment of the present invention, the plant-based product is prepared from one or more plant-based material and/or plant material. In an embodiment, the plant material is one or more legumes. In another embodiment, the plant-based material is legume slurry and/or legume milk base. In an embodiment of the present invention, the plant-based product is prepared from one or more legumes. In an embodiment, the one or more legumes is selected from soyabean, peas, chickpeas, lentils, and fava bean. In a further embodiment, the one or more legumes are legumes containing raffinose family oligosaccharides (RFOs). In a preferred embodiment, the one or more legumes is soyabean. The RFO is raffinose, stachyose and verbascose.

In an embodiment of the present invention, the plant-based product is a soydrink. In a preferred embodiment, the soydrink is soymilk.

### Alpha Qalactosidase:

In an embodiment of the present invention, the one or more alpha galactosidases is derived from Aspergillus niger. In another embodiment, the one or more alpha galactosidases hydrolyzes raffinose family oligosaccharides (RFO) to generate sucrose and galactose. The RFO is in one embodiment one or more selected from a group consisting of raffinose, stachyose and verbascose.

In an embodiment of the present invention, the one or more alpha galactosidase is polypeptide selected from the group consisting of:
(a) a polypeptide having at least 80% sequence identity to SEQ ID NO:1,
(b) a polypeptide derived from SEQ ID NO:1 by having 1-30 alterations e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions,
(c) a polypeptide derived from the polypeptide of (a), wherein the N- and/or C-terminal end has been extended by addition of one or more amino acids, and
(d) a fragment of the polypeptide of (a).

In another embodiment, the polypeptide has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO:1.

In a preferred embodiment of the present invention, the one or more alpha galactosidase is a polypeptide comprising, consisting essentially of, or consisting of SEQ ID NO: 1.

In an embodiment, the polypeptide may have an N-terminal and/or C-terminal extension of one or more amino acids, e.g., 1-5 amino acids.

In another aspect, the polypeptide is derived from SEQ ID NO: 1 by substitution, deletion or addition of one or several amino acids. In another aspect, the polypeptide is derived from a mature polypeptide of SEQ ID NO: 1 by substitution, deletion or addition of one or several amino acids. In some embodiments, the polypeptide is a variant of SEQ ID NO: 1 comprising a substitution, deletion, and/or insertion at one or more positions. In one aspect, the number of amino acid substitutions, deletions and/or insertions introduced into the polypeptide of SEQ ID NO: 1 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In one embodiment, the alpha galactosidase has the following characteristics: The alpha galactosidase is active in pH range 3-8 with an optimum around pH 3.5, application temperatures of the alpha galactosidase ranges from 4° C. to 60° C., the alpha galactosidase is inactivated at 90° C. in 10 minutes.

### Use

In one aspect, the present invention relates to use of one or more alpha galactosidases for enhancing the flavor and/or the texture of a plant-based product, wherein the plant-based product is a food and/or a drink. In an embodiment, the plant-based material is prepared from one or more legumes. The one or more legumes is selected from soyabean, peas, chickpeas, lentils, fava bean and any legumes containing raffinose family oligosaccharides. In a preferred embodiment, the one or more legumes is soyabean.

In an embodiment of the present invention, the enhanced flavor and/or texture is one or more selected from a group consisting of: improved sweetness, decreased bitterness, decreased astringency, improved richness, improved fattiness, improved sweet aftertaste, improved mouthfeel and decreased feeling of a legume/beany texture. In another embodiment, the enhanced texture is decreased feeling of a legume/beany texture.

### Method:

In an embodiment of the present invention, the method for enhancing the flavor and/or the texture of a plant-based product, the method comprises adding one or more alpha galactosidases to a plant-based material and/or the plant-based product; and incubating the plant-based product, wherein the plant-based product is a food and/or a drink.

In an embodiment, the plant-based material is used to prepare the plant-based product. In a preferred embodiment, the plant-based product is prepared from one or more legumes. The plant-based material can be legume slurry and/or legume milk base. In a preferred embodiment, the plant-based material is soy slurry and/or soymilk base. In an embodiment, the step of adding one or more alpha galactosidases to the plant-based product comprises adding 0.01% w/w to 1% w/w one or more alpha galactosidases to the plant-based product. In another embodiment, the step of adding one or more alpha galactosidase to the plant-based product comprises adding between 67 milligrams of enzyme protein per kilogram legumes (mgEP/kg) and 13.4 gram of enzyme protein per kilogram legumes (gEP/kg) of one or more alpha galactosidases to the plant-based product.

In a further embodiment, the plant-based product is incubated at a temperature range between 4 degrees Celsius (° C.) and 60° C. In another further embodiment, the plant-based product is incubated for a time period of 15 minutes to 24 hours.

In yet another embodiment, the method decreases raffinose family oligosaccharides in the plant-based product and increases sucrose and galactose in the plant-based product. In yet another embodiment, the raffinose family oligosaccharides is one or more selected from a group consisting of raffinose, stachyose and verbascose.

### Process:

In yet another aspect, the present invention relates to a process for preparing a plant-based product with enhanced flavor and/or texture, wherein the plant-based product is a food and/or a drink. The process comprises soaking one or more plant materials and grinding the one or more plant materials to a slurry. One or more alpha galactosidases is added to the slurry. Thereafter, fiber is separated from the slurry to obtain plant material base. The one or more alpha galactosidases is added to the plant material base to obtain the plant-based product.

In an embodiment, the plant material is one or more legumes. In another embodiment, the plant material base is a legume milk base.

In an embodiment, the temperature is maintained at a range of 4 degree Celsius (° C.) to 55° C. for 15 minutes to 20 hours after adding the one or more alpha galactosidases to the slurry.

In an embodiment, the temperature is maintained at a range of 4 degree Celsius (° C.) to 55° C. for 15 minutes to 20 hours after adding the one or more alpha galactosidases to the legume milk base.

In an embodiment, the method further comprises exposing the legume milk base with heat treatment to inactivate the one or more alpha galactosidases that are not inactivated.

In a preferred embodiment, a process for preparing soy-based product with enhanced flavor and/or texture comprises producing soy base and producing soymilk. The soy base production process is diverse by applying or omitting various processing steps. For producing soy base either whole or dehulled soyabeans are used. There is an option of alkaline blanching which improves the inactivation of endogenous proteases and lipoxygenases. The process comprises soaking soyabeans and grinding the soyabeans to a soy slurry. Furthermore, soaking of soyabean prior to grinding improves the grinding efficiency. The soyabeans going through a grinding process result in a colloid solution of the soyabeans in water, also called the soy slurry. The protein concentration of the soy slurry is determined by the bean to water ratio at the inlet to the grinders. One or more alpha galactosidases is added to the soy slurry. Thereafter, fiber is separated from the soy slurry to obtain soymilk base. The one or more alpha galactosidases is added to the soymilk base to obtain the soy-based product.

In the soy slurry, one or more alpha galactosidases is added. The temperatures to be applied is between 4° C. and 55° C. from 15 minutes to 20 hours.

In the next step the fiber particles, which are called okara, are separated by using a decanter or centrifuge. The efficiency of the decanter determines the yield as well as the quality of the soymilk. For optimized yield, the moisture content in the okara should be low. The amount of sediment (dry matter) in the slurry should be low to avoid chalkiness caused by insoluble particles in the soy-based product.

For removal of undesirable volatile off-flavors giving beany flavor, the soy base is exposed to a heat treatment, such as direct steam infusion which heats to 140° C. followed by flash cooling. The heat treatment inactivates any endogenous or added enzyme such as alpha galactosidase not yet inactivated.

The soy base is standardized to the desired protein content and composition by adding water and other ingredients and further processing into soy-based product such as soymilk, soy drink, soy yoghurt and soy ice cream.

In the standardized soy base, different formulations can be added. The final soymilk looks different dependent on the region or type of soyabeans. Soymilk typically contains around 3% protein, 2% oil and 2% carbohydrates. A soymilk can also be a beverage with a reduced protein content but higher sugar level. Soymilk beverages can be mineral fortified or flavored.

In another preferred embodiment, the one or more alpha galactosidases is added to the soy base. The incubation temperature ranges from 4° C. to 55° C. and the incubation time ranges from 15 minutes to 20 hours.

The formulated soy base is then homogenized, and the soymilk is exposed to an ultra-heat treated (UHT) process and aseptic filling, thereby obtaining a long shelf-life for the soymilk at room temperature. The UHT process applied can be direct or indirect. In the direct UFT process, the steam is briefly injected into a product to be treated, and rapidly followed by flash cooling. In indirect UHT process, a product to be treated does not come into direct contact with the heat source but is instead heated using heat exchangers. UHT process inactivates any added enzyme such as alpha galactosidases in the soymilk.

Particular embodiments of the present disclosure are described in the following numbered paragraphs:
1. Use of one or more alpha galactosidases for enhancing the flavor and/or the texture of a plant-based product, wherein the plant-based product is a food and/or a drink.
2. The use according to paragraph 1, wherein the one or more alpha galactosidase is derived from Aspergillus niger.
3. The use according to paragraphs 1 or 2, wherein the one or more alpha galactosidase hydrolyzes raffinose family oligosaccharides (RFO) to generate sucrose and galactose.
4. The use according to paragraph 3, wherein the RFO is one or more selected from a group consisting of raffinose, stachyose and verbascose.
5. The use according to any one of the preceding paragraphs, wherein the one or more alpha galactosidase is a polypeptide selected from a group consisting of:
   (a) a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
   (b) a polypeptide derived from SEQ ID NO:1 by having 1-30 alterations e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a polypeptide derived from the polypeptide of (a), wherein the N- and/or C-terminal end has been extended by addition of one or more amino acids; and
   (d) a fragment of the polypeptide of (a).
6. The use according to paragraphs any one of the preceding paragraphs, wherein the one or more alpha galactosidases is a polypeptide having sequence of SEQ ID NO: 1.
7. The use according to any of the preceding paragraphs, wherein the enhanced flavor and/or texture is one or more selected from a group consisting of: improved sweetness, decreased bitterness, decreased astringency, improved richness, improved fattiness, improved sweet aftertaste, improved mouthfeel, and decreased feeling of a legume/beany texture.
8. The use according to any of the preceding paragraphs, wherein the enhanced texture is decreased feeling of a legume/beany texture.
9. The use according to any of the preceding paragraphs, wherein the plant-based product is prepared from one or more legumes.
10. The use according to any of the preceding paragraphs, wherein the one or more legumes is selected from soyabean, peas, chickpeas, lentils, fava bean and any legumes containing raffinose family oligosaccharides.
11. The use according to any of the preceding paragraphs, wherein the one or more legumes is soyabean.
12. The use according to any of preceding paragraphs, wherein the plant-based product is a soydrink.
13. The use according to paragraph 12, wherein the soydrink is soymilk.
14. A method for enhancing the flavor and/or the texture of a plant-based product, the method comprising:
   a. adding one or more alpha galactosidases to a plant-based material and/or the plant-based product; and
   b. incubating the plant-based material and/or the plant-based product, wherein the plant-based product is a food and/or a drink.
15. The method according to paragraphs 14, wherein the one or more alpha galactosidases is derived from Aspergillus niger.
16. The method according to paragraph 14 or 15, wherein the one or more alpha galactosidase hydrolyzes raffinose family oligosaccharides (RFO) to generate sucrose and galactose.
17. The method according to any one of paragraphs 14-16, wherein the one or more alpha galactosidase is a polypeptide selected from a group consisting of:
   (a) a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
   (b) a polypeptide derived from SEQ ID NO:1 by having 1-30 alterations e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a polypeptide derived from the polypeptide of (a), wherein the N- and/or C-terminal end has been extended by addition of one or more amino acids; and
   (d) a fragment of the polypeptide of (a).
18. The method according to paragraph 14-17, wherein the one or more alpha galactosidases is a polypeptide having sequence of SEQ ID NO: 1.
19. The method according to paragraph 14-18, wherein the plant-based material is used to prepare the plant-based product.
20. The method according to any one of paragraphs 14-19, wherein the enhanced flavor and/or texture is selected from a group consisting of: improved sweetness, decreased bitterness, decreases astringency, improved richness, improves fattiness, improved sweet aftertaste, improved mouthfeel and decreased feeling of a legume/beany texture.
21. The method according to paragraph 20, wherein the enhanced texture is legume/beany texture.
22. The method according to any one of paragraphs 14-21, wherein the plant-based product is prepared from one or more legumes.
23. The method according to paragraph 14-22, wherein the plant-based material is legume slurry and/or legume milk base.
24. The method according to paragraphs 22, wherein the one or more legumes is selected from soyabean, peas, chickpeas, lentils, fava beans and any legumes containing raffinose family oligosaccharides.
25. The method according to paragraphs 22, wherein the one or more legumes is soyabean.
26. The method according to paragraph 14-25, wherein the plant-based material is soy slurry and/or soymilk base.
27. The method according to any one of paragraphs 14-26, wherein the plant-based product is a soydrink.
28. The method according to paragraphs 27, wherein the soydrink is soymilk.
29. The method according to any one of paragraphs 14-28, wherein the method comprises 0.01% w/w to 1% w/w one or more alpha galactosidases to the plant-based product.
30. The method according to any one of paragraphs 14-29, wherein the method comprises adding between 67 mgEP/kg and 13.4 gEP/kg of one or more alpha galactosidases to the plant-based product.
31. The method according to any one of paragraphs 14-30, wherein the plant-based product is incubated at a temperature range between 4 degrees Celsius (° C.) and 60° C.
32. The method according to any one of paragraphs 14-31, wherein the plant-based product is incubated for a time period of 15 minutes to 24 hours.
33. The method according to any one of paragraphs 14-32, wherein the method decreases raffinose family oligosaccharides in the plant-based product and increases sucrose and galactose in the plant-based product.
34. A process for preparing a plant-based product with enhanced flavor and/or texture, the process comprising:
   a. soaking one or more plant materials;
   b. grinding the one or more plant materials to a slurry;
   c. adding one or more alpha galactosidases to the slurry;
   d. separating fiber from the slurry to obtain plant material base;
   e. adding one or more alpha galactosidases to the plant material base
      wherein the plant-based product is a food and/or a drink.
35. The process for preparing the plant-based product with enhanced flavor and/or texture according to paragraph 34, wherein the temperature is maintained at a range of 4 degree Celsius (° C.) to 55° C. for 15 minutes to 20 hours after adding the one or more alpha galactosidases to the slurry.
36. The process for preparing the plant-based product with enhanced flavor and/or texture according to paragraph 34 or 35, wherein further comprises exposing the legume milk base with heat treatment to inactivate the one or more alpha galactosidases that are not inactivated.
37. The process for preparing the plant-based product with enhanced flavor and/or texture according to any one of paragraphs 34-36, wherein the temperature is maintained at a range of 4 degree Celsius (° C.) to 55° C. for 15 minutes to 20 hours after adding the one or more alpha galactosidases to the legume milk base.
38. The process according to any one of paragraphs 34-37, wherein the one or more alpha galactosidase is derived from Aspergillus niger.
39. The process according to any one of paragraphs 34-38, wherein the one or more alpha galactosidase hydrolyses raffinose family oligosaccharides (RFO) to generate sucrose and galactose.
40. The process according to paragraphs 34-39, wherein the RFO is selected from a group consisting of raffinose, stachyose and verbascose.
41. The process according to any one of paragraphs 34-40, wherein the one or more alpha galactosidase is a polypeptide selected from a group consisting of:
   (a) a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
   (b) a polypeptide derived from SEQ ID NO:1 by having 1-30 alterations e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (c) a polypeptide derived from the polypeptide of (a), wherein the N- and/or C-terminal end has been extended by addition of one or more amino acids; and
   (d) a fragment of the polypeptide of (a).
42. The process according to paragraph 34-41, wherein the one or more alpha galactosidases is a polypeptide having sequence of SEQ ID NO: 1.
43. The process according to any one of paragraphs 34-42, wherein the enhanced flavor and/or texture is selected from a group consisting of: improved sweetness, decreased bitterness, decreases astringency, improved richness, improves fattiness, improved sweet aftertaste, improved mouthfeel and decreased feeling of a legume/beany texture.
44. The process according to paragraph 43, wherein the enhanced texture is legume/beany texture.
45. The process according to any one of paragraphs 34-44, wherein the plant material is one or more legumes.
46. The process according to any one of paragraphs 34-45, wherein the one or more legumes is selected from soyabean, peas, chickpeas, lentils, fava bean and any legumes containing raffinose family oligosaccharides.
47. The process according to paragraph 45, wherein the one or more legumes is soyabean.
48. The process according to paragraph 34-47, wherein the plant material base is a legume milk base.
49. The process according to paragraph 48, wherein the legume milk base is soymilk base.
50. The process according to any one of paragraphs 34-49, wherein the plant-based product is a soydrink.
51. The process according to paragraph 50, wherein the soydrink is soymilk.

### EXAMPLES

The following examples are not intended to be a detailed catalogue of all the different ways in which the present disclosure may be implemented or of all the features that may be added to the present disclosure. Subjects skilled in the art will appreciate that numerous variations and additions to the various embodiments may be made without departing from the present disclosure. Hence, the following descriptions are intended to illustrate some particular embodiments of the invention and not to exhaustively specify all permutations, combinations and variations thereof.

Unless otherwise indicated, the percentages set forth in the following examples are by weight, based upon the total weight of the composition.

### Material and Methods

The experiments were performed in Denmark with commercially available soymilk from Nature Green and alpha galactosidase having SEQ ID NO:1.

### Example 1—Sensory Analysis of Alpha Galactosidase Treated Soymilk and Untreated Soymilk with 22 Untrained Assessors

### A. Sample Production

In the trial, a commercial soymilk from Natur Green was used as a substrate for alpha galactosidase incubation. Incubation was conducted in the Thermomix Vorwerk. The soymilk was briefly preheated to 40° C.; the alpha galactosidase was added at a dosage of 0.013 mgEP/1 mL soymilk. Furthermore, a blank sample without alpha galactosidase was also carried out in parallel. Incubation was carried out for 1 hour at 40° C. After the incubation, the Vorwerk temperature was increased to 90° C. to inactivate enzymes and holding time was 10 minutes. The soymilk was cooled in an ice batch and transferred into a pasteurized glass bottle and stored at 4° C. until sensory analysis.

### B. Sugar Analysis

The treated soymilk samples were analyzed on HPLC (High pressure liquid chromatography) with refractive index detection. The column used was an Aminex HPX-87P column (from Bio-Rad) placed in a column oven set to 80° C. The separation was carried using milliQ water at an isocratic level. The detection was carried using a Reflective Index (RI) detector (Thermo Scientific™ RefractoMax 521 Refractive Index Detector). The flow was 0.6 milliliter per minute (mL/min). The chemical pure standards of raffinose, stachyose, galactose and sucrose were used for calibration. Prior to HPLC analysis, 5 microliter (μL) of a 50% acetic acid was added to the soymilk samples; the samples were centrifuged for 5 minutes at 14000 rpm and 10 minutes. Afterwards, sample clean-up was continued by adding 5 μL Carrez 1 and 5 μL Carrez 2 and spinning down the samples at the above-mentioned conditions. Finally, the samples were filtered through a 0.22 μm filter.

TABLE 1
Results of HPLC analysis

| | Mean | Std Dev | Mean | Std Dev | | Std Dev | Mean | Std Dev |
|---|---|---|---|---|---|---|---|---|
| Sample | (Stachyose | (stachyose | (Raffinose | (Raffinose | Mean | (Sucrose | (Galactose | (Galactose |
| Name | gL) | gL) | gL) | gL) | (sociolegal) | gL) | gL) | g per L) |
| Soymilk | 0.12 | 0.01 | 0.13 | 0.01 | 9.67 | 0.35 | 2.92 | 0.09 |
| with alpha | | | | | | | | |
| galactosidase | | | | | | | | |
| Soymilk | 4.65 | 0.01 | 2.32 | 0.02 | 6.52 | 0.01 | 0.25 | 0 |
| blank | | | | | | | | |
| Soymilk | 4.66 | 0.26 | 2.31 | 0.11 | 6.49 | 0.32 | 0.25 | 0.01 |
| unprocessed | | | | | | | | |

As shown in Table 1, all raffinose and stachyose were hydrolyzed by the alpha galactosidases to sucrose and galactose. An increase in sucrose from 6.5 g per L to 9.6 g per L was measured. Galactose was increasing from 0.25 gL to 2.9 g per L.

### C. Sensory Analysis

A paired comparison focused on sweetness was performed with 22 untrained assessors. Assessors were asked to identify the sweetest sample. Furthermore, they were asked to provide details on other differences between the samples in a comment field. Sensory analysis was paper based. Samples were served at room temperature in transparent plastic cups with 3-digit labels. Each assessor received a 30 ml (about 1.01 oz) sample.

### Results

20 out of 22 people recognized the 0.02% alpha galactosidase treated soymilk (0.013 mgEP/1 mL soymilk) as sweetest. The alpha galactosidase had a significant impact on the sensory regarding sweetness (binomial test). Assessors also added comments on the paper while doing the sensory test. Alpha galactosidase treated soymilk was found more fatty, richer, and having more body.

### Example 2—Two Round Sensory Analysis of Alpha Galactosidase Treated Soymilk and Untreated Soymilk with 6 Untrained Assessors

### A. Sample Production

Same production method as described in Example 1

### B. Sugar Analysis

Same sugar analysis method as described in Example 1

TABLE 2
Results of HPLC analysis

| | Mean | Std Dev | Mean | Std Dev | Mean | Std Dev | Mean | Std Dev |
|---|---|---|---|---|---|---|---|---|
| sample | (stachyose | (stachyose | (raffinose | (raffinose | (sucrose | (sucrose | (galactose | galactose |
| name | gL) | gL) | gL) | gL) | gL) | gL) | gL) | gL) |
| soymilk alpha | 0.09 | 0 | 0.2 | 0.01 | 6.72 | 0.24 | 2.99 | 0.09 |
| galactosidase | | | | | | | | |
| soymilk blank | 6.12 | 0.04 | 1.95 | 0 | 4.79 | 0.05 | 0.21 | 0.01 |
| soymilk | 6.11 | 0.22 | 1.94 | 0.04 | 4.77 | 0.18 | 0.21 | 0 |
| unprocessed | | | | | | | | |

As shown in Table 2, all raffinose and stachyose were hydrolyzed by the alpha galactosidases to sucrose and galactose. An increase in sucrose from 4.8 g per L to 6.7 g per L was measured. Galactose was increasing from 0.2 gL to 3 g per L.

### C. Sensory Analysis

In this sensory analysis, 6 untrained assessors were in the sensory panel. Sensory trials were performed in 2 rounds:

Round 1: A training session was carried out where assessors drank the samples (blank and 0.013 mgEP/1 mL soymilk) and identified the attributes to score: sweetness, bean taste and full-body perception. During the training, a 9-point hedonic scale was used. The sample size was 50 ml.

Round 2: Blind tasting coupled with scoring was carried out. A paper-based 9-point hedonic scale was used by the assessors. A sample size of 30 ml (about 1.01 oz) was provided. Samples were served at room temperature in transparent plastic cups with 3-digit labels.

As shown in Table 3, the sweetness generated from the alpha galactosidase was significantly noticed and were scored significantly (p<0.05) by the assessors.

TABLE 3

| Sample_ID | Sweetness scoring |
|---|---|
| blank | 3 |
| Alpha galactosidase_0.02 pct | 4.7 |

When looking at the full-body and beaniness attribute scoring—4 assessors were able to distinguish between a blank and an alpha galactosidase treated sample of soymilk. There is a tendency of assessors to incline towards alpha galactosidase treated sample of soymilk.

### Example 3—Sensory Analysis of Alpha Galactosidase Treated Soymilk and Untreated Soymilk with 23 Untrained Assessors

### A. Sample Production

Same production method as described in Example 1

### B. Sugar Analysis

Same sugar analysis method as described in Example 1

TABLE 4
Results of HPLC analysis

| | Mean | Std Dev | Mean | Std Dev | Mean | Std Dev | Mean | Std Dev |
|---|---|---|---|---|---|---|---|---|
| Sample | (stachyose | (stachyose | (raffinose | (raffinose | (sucrose | (sucrose | (galactose | (galactose |
| name | gL) | gL) | gL) | gL) | gL) | gL) | gL) | gL) |
| soymilk alpha | 0.22 | 0 | 0.13 | 0 | 9.23 | 0.25 | 2.49 | 0.02 |
| galactosidase | | | | | | | | |
| soymilk blank | 4.09 | 0.04 | 1.75 | 0.03 | 6.93 | 0.07 | 0.08 | 0.01 |
| soymilk | 3.71 | 0.05 | 1.61 | 0.09 | 6.37 | 0.15 | 0.06 | 0.01 |
| unprocessed | | | | | | | | |

As shown in Table 4, all raffinose and stachyose are hydrolyzed by the alpha galactosidase to sucrose and galactose. An increase in sucrose from 6.9 g per L to 9.2 g per L was measured. The galactose was increasing from 0.08 gL to 2.5 g per L.

### C. Sensory Analysis

### Objective:

The objective of the sensory evaluation was to investigate if there is a difference in sweetness between the two soymilk samples (with/without alpha galactosidase). Furthermore, other differences that exists between the samples were also evaluated.

### Method:

A paired comparison focused on sweetness was performed with 23 untrained assessors. For the assessors to focus better on the low sweetness level, nose clips were provided which reduce the influence of aroma and allow focus on taste and mouthfeel. The assessors were asked to identify the sweetness sample but were also given the possibility to rate both samples as equal. Furthermore, the assessors were asked to provide details on other differences between the samples in a comment field. The data was collected using the sensory software EyeQuestion.

The samples were served at approximately 10° C. in transparent plastic cups with 3-digit labels. Each assessor received a 30 ml (about 1.01 oz) sample.

### Results:

The comments of the assessors who identified the sample with alpha galactosidase as the sweetest showed that there might also be a difference in mouthfeel since the sample with alpha galactosidase was described as slightly creamier/fuller/rounder.

Table 5 shows the results of the paired comparison. Most of the assessors selected the sample with alpha galactosidase as the sweetest sample. Based on the p-value of 0.077, there is a significant difference in sweetness between the soymilk with and without alpha galactosidase was concluded.

TABLE 5

| Sweetest sample | No. of assessors |
|---|---|
| No difference | 7 |
| With alpha galactosidase | 12 |
| Without alpha galactosidase | 4 |

1-8. (canceled)

9. A method for enhancing the flavor and/or the texture of a plant-based product, the method comprising:
a. adding one or more alpha galactosidases to a plant-based material and/or the plant-based product; and
b. incubating the plant-based material and/or the plant-based product,
wherein the plant-based product is a food and/or a drink.

10. The method according to claim 9, wherein the one or more alpha galactosidase is a polypeptide selected from a group consisting of:
(a) a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1;
(b) a polypeptide derived from SEQ ID NO:1 by having 1-30 alterations e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions; and
(c) a polypeptide derived from the polypeptide of (a), wherein the N- and/or C-terminal end has been extended by addition of one or more amino acids.

11. The method according to claim 9, wherein the one or more alpha galactosidases is a polypeptide having sequence of SEQ ID NO: 1.

12. The method according to claim 9, wherein the method comprises 0.01% w/w to 1% w/w one or more alpha galactosidases to the plant-based product.

13. The method according to claim 9, wherein the method comprises adding between 67 mgEP/kg and 13.4 gEP/kg of one or more alpha galactosidases to the plant-based product.

14. A process for preparing a plant-based product with enhanced flavor and/or texture, the process comprising:
a. soaking one or more plant materials;
b. grinding the one or more plant materials to a slurry;
c. adding one or more alpha galactosidases to the slurry;
d. separating fiber from the slurry to obtain plant material base; and
e. adding one or more alpha galactosidases to the plant material base,
wherein the plant-based product is a food and/or a drink.

15. The process according to claim 14, wherein the plant material base is a legume milk base.

16. The process according to claim 14, wherein the plant material base is a soy milk base.

17. The method according to claim 9, wherein the one or more alpha galactosidases is a polypeptide having sequence with at least 95% sequence identity to SEQ ID NO: 1.

18. The method according to claim 9, wherein the enhanced flavor and/or texture of the plant-based product is improved sweetness, decreased bitterness, decreased astringency, improved richness, improved fattiness, improved sweet aftertaste, and/or improved mouthfeel.

19. The method according to claim 9, wherein the plant based material and/or the plant-based product is prepared from one or more legumes.

20. The method according to claim 19, wherein the one or more legumes is soyabean.

21. The method according to claim 19, wherein the enhanced flavor and/or texture is one or more selected from a group consisting of: improved sweetness, decreased bitterness, decreased astringency, improved richness, improved fattiness, improved sweet aftertaste, improved mouthfeel, and decreased feeling of a legume/beany texture.

22. The method according to claim 9, wherein the plant-based product is a soy drink.

23. The method according to claim 9, wherein the plant-based product is soy milk.


SEQUENCE LISTING (1 sequence)

SEQ ID NO: 1 - protein, 725 aa, Aspergillus niger
APATGASNSQTIVTNGTSFALNGDNVSYRFHVNSTTGDLISDHFGGVVSGTIPSPVEPAVNGWVGMPGRIRREFPDQGRGDFRIPAVRIRESAGYTVSDLQYVSHEVIEGKNALPGLPATFGDAQAVTTLVVHLYDNYSSVAADLSYSIFPKYDAIVRSVNVINQGPGNITIEALASISIDFPYEDLDMVSLRGDWAREANVQRSKVQYGVQGFGSSTGYSSHLHNPFLAIVDPATTESQGEAWGFNLVYTGSFSAQVEKGSQGFTRALLGFNPDQLSWNLGPGETLTSPECVAVYSDKGLGSVSRKFHRLYRNHLMKSKFATSDRPVLLNSWEGVYFDYNQSSIETLAEESAALGVHLFVMDDGWFGDKYPRVSDNAGLGDWMPNPARFPDGLTPVVQDITNLTVNGTESTKLRFGIWVEPEMVNPNSTLYHEHPEWALHAGPYPRTERRNQLVLNLALPAVQDFIIDFMTNLLQDTGISYVKWDNNRGIHETPSPSTDHQYMLGLYRVFDTLTTRFPDVLWEGCASGGGRFDAGMLQYVPQIWTSDNTDAIDRITIQFGTSLAYPPSAMGAHLSAVPNAQTGRTVPITFRAHVAMMGGSFGLELDPATVEGDEIVPELLALAEKVNPIILNGDLYRLRLPQDSQWPAALFVTQDGAQAVLFYFQVQPNVNHAVPWVRLQGLDPKADYTVDGDQTYSGATLMNLGLQYSFDTEYGSKVVFLERQ